(19) United States
(12) Patent Application Publication
(10) Pub. No.: US 2024/0058619 A1
(43) Pub. Date: Feb. 22, 2024

(54) IMPLANTABLE PHOTOBIOMODULATION SYSTEMS EMPLOYING THERMAL MONITORING OR CONTROL AND METHODS OF MAKING AND USING

(71) Applicant: Boston Scientific Neuromodulation Corporation, Valencia, CA (US)

(72) Inventors: Changfang Zhu, Valencia, CA (US); Rafael Carbunaru, Valley Village, CA (US)

(21) Appl. No.: 18/232,649

(22) Filed: Aug. 10, 2023

Related U.S. Application Data
(60) Provisional application No. 63/399,982, filed on Aug. 22, 2022.

(51) Int. Cl. A61N 5/06

(52) U.S. Cl. CPC A61N 5/0601; A61N 2005/0626; A61N 2005/063

(57) ABSTRACT

A method for photobiomodulation of tissue includes emitting light from a lead implanted in the tissue using an implanted light source according to a first delivery program; repeatedly estimating an amount, speed, or time of a temperature or temperature change of, or amount of heat generated by, the implanted light source or repeatedly estimating an amount, speed, or time of a temperature or temperature change of tissue receiving the emitted light; and when the estimate exceeds a first threshold value and the light is emitted according to the first delivery program, emitting light from the implanted lead using the implanted light source according to a second delivery program, wherein the second delivery program results in lower heat generation by the implanted light source over a period of time than the first delivery program.

### CROSS-REFERENCE TO RELATED APPLICATIONS

This application claims the benefit under 35 U.S.C. § 119(e) of U.S. Provisional patent application Ser. No. 63/399,982, filed Aug. 22, 2022, which is incorporated herein by reference.

### FIELD

The present disclosure is directed to the area of implantable photobiomodulation (PBM) or PBM/electrical stimulation systems and methods of making and using the systems. The present disclosure is also directed to implantable PBM or PBM/electrical stimulation systems that include thermal monitoring or thermal control.

### BACKGROUND

Implantable neuromodulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

Photobiomodulation (PBM) can also provide therapeutic benefits in a variety of diseases and disorders by itself or in combination with electrical stimulation. An PBM system may include one or more light sources and, often, one or more optical fibers to carry the light to the desired modulation site.

### BRIEF SUMMARY

In one aspect, a method for photobiomodulation of tissue includes emitting light from a lead implanted in the tissue using an implanted light source according to a first delivery program; repeatedly estimating an amount, speed, or time of any one, or any combination of, a temperature or a temperature change of, or amount of heat generated by, the implanted light source or repeatedly estimating an amount, speed, or time of any one, or any combination of, a temperature or a temperature change of tissue receiving the emitted light; and when the estimate exceeds a first threshold value and the light is emitted according to the first delivery program, emitting light from the implanted lead using the implanted light source according to a second delivery program, wherein the second delivery program results in lower heat generation by the implanted light source over a period of time than the first delivery program.

In another aspect, a method for photobiomodulation of tissue includes emitting light from a lead implanted in the tissue using an implanted light source according to a first delivery program; repeatedly estimating an amount, speed, or time of any one, or any combination of, a temperature or a temperature change of, or amount of heat generated by, the implanted light source or repeatedly estimating an amount, speed, or time of any one, or any combination of, a temperature or a temperature change of tissue receiving the emitted light; when the estimate exceeds a first threshold value and the light is emitted according to the first delivery program, requesting user confirmation to switch to a second delivery program; and after receiving the user confirmation, emitting light from the implanted lead using the implanted light source according to the second delivery program, wherein the second delivery program results in lower heat generation by the implanted light source over a period of time than the first delivery program.

In at least some aspects, the method further includes, when the estimate falls below a second threshold value and the light is emitted according to the second delivery program, emitting light from the implanted lead using the implanted light source according to the first delivery program. In at least some aspects, the first threshold value and the second threshold value are different.

In at least some aspects, the estimating includes making a measurement to estimate repeatedly estimating an amount, speed, or time of any one, or any combination of, a temperature or a temperature change of, or amount of heat generated. In at least some aspects, making the measurement includes making the measurement regularly with a specified periodicity. In at least some aspects, making the measurement includes making a temperature measurement using a thermocouple, a thermistor, a resistance thermal detector (RTD), infrared sensor, or an integrated circuit thermal sensor. In at least some aspects, making the measurement includes making an electrical measurement of the light source. In at least some aspects, making the electrical measurement includes making a measurement of current, voltage, or impedance, or of a change in current, voltage, or impedance, of the light source. In at least some aspects, making the measurement includes making a measurement of the light emitted by the light source.

In yet another aspect, a system for photobiomodulation of tissue includes a light source; an implantable lead including a distal region and a light emitter disposed along the distal region, wherein the light emitter is either the light source or coupled to the light source by at least one optical waveguide so that light from the light source is emitted from the light emitter; and an implantable control module coupled to the light source for directing the light source to generated light, the implantable control module includes a memory storing instructions and a processor configured to execute the instructions, the instructions embodying any of the methods presented above.

In a further aspect, a photobiomodulation system includes a programmer having a processor configured for programming of an implantable control unit for generating delivery program for an implanted light source, the processor configured to perform actions including: receiving a selection of a value for each of a plurality of delivery parameters, wherein the selection of the value for each of the delivery parameters is limited by expected heat generation or temperature change arising from the selection of the value and any previously selected values; and, after the selection of all of the values, estimating expected heat generation and, when the expected heat generation or temperature change exceeds a threshold either i) providing a warning or recommendation to a programmer or ii) requiring the programmer to alter at least one of the selected values.

In yet another aspect, a photobiomodulation system includes an implantable light source; an implantable lead including a distal region and a light emitter disposed along the distal region, wherein the light emitter is either the implantable light source or coupled to the implantable light source by at least one optical waveguide so that light from the implantable light source is emitted from the light emitter into tissue; a thermoelectric cooling device coupled to the implantable light source and configured for removing heat generated by the implantable light source; and an implantable control module coupled to the implantable light source for directing the implantable light source to generated light and coupled to the thermoelectric cooling device for directing the thermoelectric cooling device to remove heat generated by the implantable light source.

In at least some aspects, the implantable light source and the thermoelectric cooling device are disposed in the implantable lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of a photobiomodulation (PBM) or PBM/electrical stimulation system with a control module and a lead having a light emitter;

FIG. 2 is a schematic side view of a control module and a portion of a lead of another embodiment of a photobiomodulation (PBM) or PBM/electrical stimulation system;

FIG. 3 is a schematic side view of a portion of another embodiment of a lead with multiple light emitters;

FIG. 4 is a schematic side view of a control module and a portion of a lead of a yet another embodiment of a photobiomodulation (PBM) or PBM/electrical stimulation system;

FIG. 5 is a schematic side view of a control module, a lead extension, and a portion of a lead of a further embodiment of a photobiomodulation (PBM) or PBM/electrical stimulation system;

FIG. 6 is an example of a graph of voltage versus current for a light source at different temperatures;

FIG. 7 is an example of a graph of dV/dI versus current for a light source at different temperatures;

FIG. 8 is an example of a graph of threshold current versus temperature for a light source;

FIG. 9 is an example of a graph of dV/dI versus temperature for a light source;

FIG. 10 is a flowchart of one embodiment of a method for generating a delivery program;

FIG. 11 is a flowchart of one embodiment of a method for photobiomodulation of tissue; and

FIG. 12 is a block diagram of one embodiment of a system for PBM or PBM/electrical stimulation.

### DETAILED DESCRIPTION

The present disclosure is directed to the area of implantable photobiomodulation (PBM) or PBM/electrical stimulation systems and methods of making and using the systems. The present disclosure is also directed to implantable PBM or PBM/electrical stimulation systems that include thermal monitoring or thermal control.

The systems described herein can produce PBM or both PBM and electrical stimulation. In at least some of these embodiments, PBM can be provided through a modification of an electrical stimulation system. PBM may include, but is not necessarily limited to, optical modulation, other modulation, optical stimulation, or any other effects resulting from response to particular wavelengths or wavelength ranges of light or from thermal effects generated using light or from any combination thereof. It will be recognized that the thermal monitoring and thermal control arrangements and methods described herein can also be applied to implantable systems for oximetry, spectroscopy, or the like or any combination of these system and photobiomodulation systems.

An implantable PBM or PBM/electrical stimulation system includes at least one light source, such as a light emitting diode (LED), light emitting transistor (LET), laser diode, a vertical cavity side-emitting laser (VCSEL), an organic light emitting diode (OLED), an organic light emitting transistor (OLET), a lamp, or any other suitable light source. The light source can be used to deliver light in pulses or in a continuous wave (CW) mode, or any combination thereof.

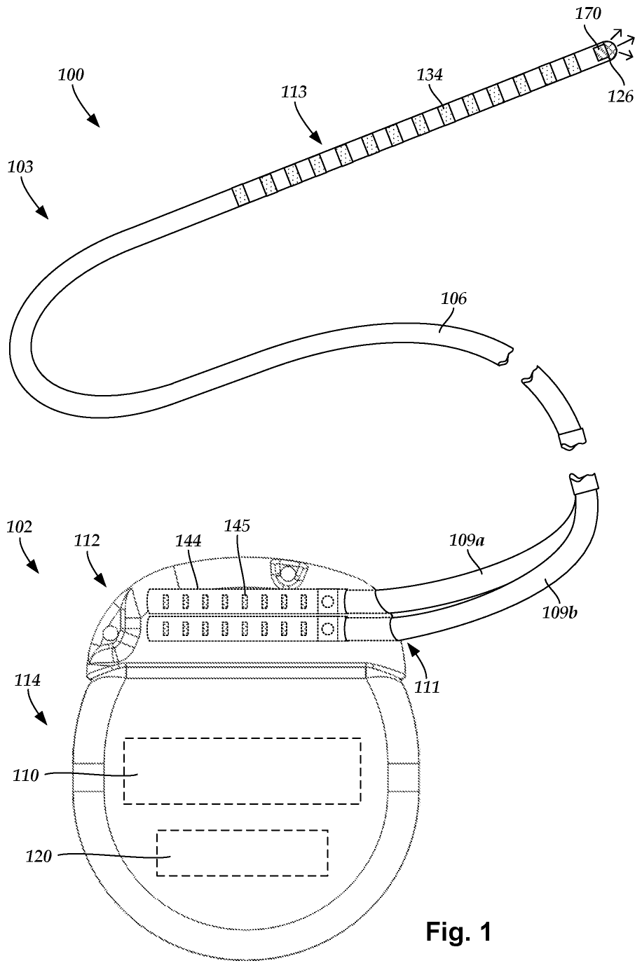

FIG. 1 is a schematic side view of a portion of an embodiment of an PBM or PBM/electrical stimulation system 100 that includes a control module 102 and a lead 103. The control module includes a sealed electronics housing 114 with an electronic subassembly 110 and an optional power source 120. The control module also includes connector housing 112 (which is also often called a header) that houses a control module connector 144 that defines at least one port 111 into which a proximal end 109a, 109b of the lead 103 can be inserted. The control module connector 144 also includes connector contacts 145 disposed within each port 111. The control module 102 (or other device) can define any suitable number of ports including, for example, one, two, three, four, five, six, seven, eight, or more ports. In FIG. 1, the lead 102 is shown coupled into a two ports 111 defined in the control module connector 112. Other embodiments of a control module 102 may have more or fewer components.

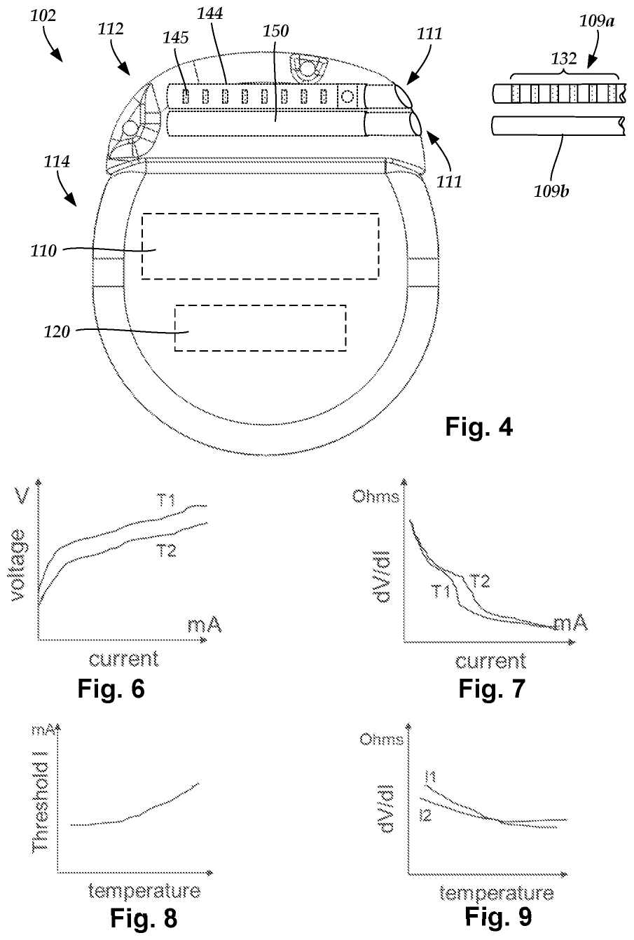

When the proximal end 109a, 109b of the lead 102 is inserted into the port 111, the connector contacts 145 can be aligned with a plurality of terminals 132 (FIG. 4) disposed along the proximal end(s) of the lead. Examples of connectors in control modules are found in, for example, U.S. Pat. Nos. 7,244,150 and 8,224,450, which are incorporated herein by reference in their entireties, as well as other references listed herein.

The optional power source 112 can provide power to the electronic subassembly 110. The electronic subassembly 110 is, at least in some embodiments, programmable and is configured to direct the PBM and, if present, electrical stimulation. The electronic subassembly 110 is electrically coupled to the connector contacts 144 and controls the light source 150. In at least some embodiments, when the light source 150 is remote from the control module 102, the electronic subassembly 110 can control the light source through signals sent to the connector contacts 144 and through the terminals 168 and conductors of the lead 166 to the light source. The electrodes 134 can be ring electrodes, tip electrodes, segmented electrodes 135 (FIG. 3), or any combination thereof.

The lead 103 includes a lead body 106, one or more proximal ends 109a, 109b, one or more distal ends 113, at least one light emitter 126 disposed along the distal end, one or more optional electrodes 134 disposed along the distal end, and one or more optional terminals 132 (FIG. 4) disposed along the proximal end of the lead. Conductors (not shown) extend from the terminals 132 to the optional electrodes 134.

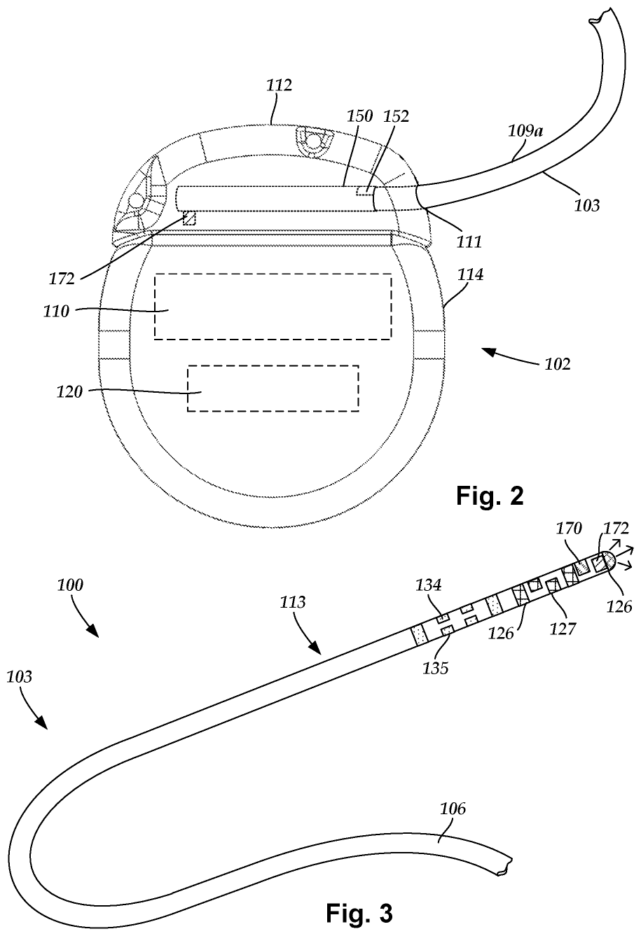

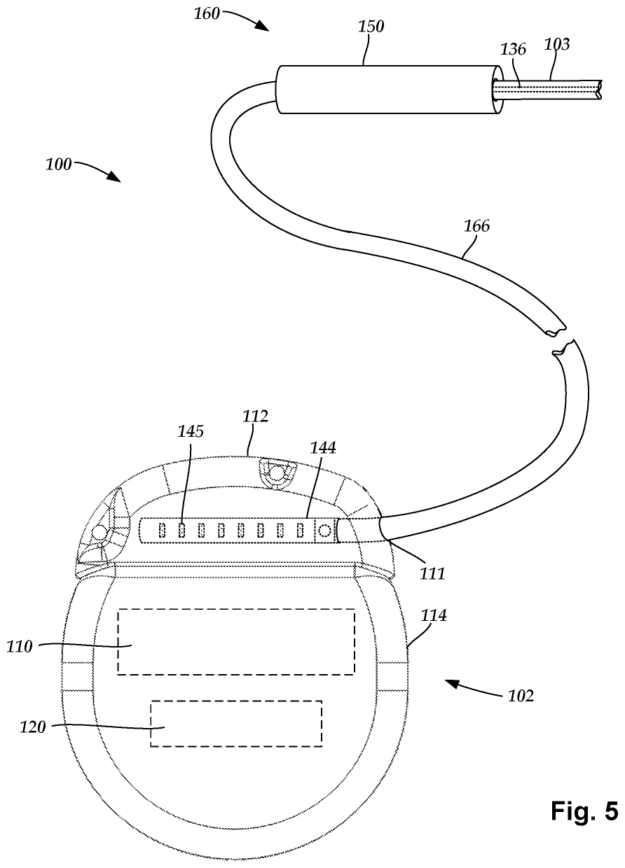

The light emitter 126 can be a light source (similar to light source 150 of FIGS. 2 to 4) or can be a light emission region of an optical waveguide 136 (FIG. 5) or the like. When the light emitter 126 is a light source, conductors (not shown) extend from the terminals 132 (FIG. 4) to the light emitter to power and operate the light source. Examples of such a light emitter can be found in U.S. Pat. No. 10,335,607, incorporated herein by reference in its entirety.

When the light emitter 126 is a light emission region, the light source can be disposed in the control module 102, lead 103, or other components as described below. Light from the light source is transmitted along one or more optical waveguides 136 (FIG. 5) or the like to the light emission region.

FIG. 2 illustrates one embodiment of a control module 102 with a light source 150 and associated optics disposed in the connector housing 112. The light source 150 provides light to a waveguide 136 (FIG. 5) in the lead 103. FIG. 4 illustrates another embodiment of a control module 102 and lead 103 in which one proximal end 109a of the lead 103 includes terminals 132 that couple to electrodes 134 (FIG. 1) on the lead and a second proximal end 109b of the lead coupled to a light source 150 in the connector housing 112. It will be understood that, as an alternative to the embodiments in FIG. 2 or FIG. 4, the light source 150 can be disposed in the sealed electronics housing 114 with a waveguide extending out of the sealed electronics housing for coupling to waveguide 136 (FIG. 5) in the lead 103.

FIG. 5 illustrate a further embodiment of a control module 102 and a lead 102 that also includes a lead extension 160 with a lead extension body 166 that includes terminals (like the terminals 132 of FIG. 4) on a proximal end for coupling to the control module connector 114 in the connector housing 112. The lead extension 160 includes a light source 150 disposed on a distal end of the lead extension and is configured to receive a proximal end of the lead 103 and deliver light into a waveguide 136 in the lead. The waveguide 136 can be a fiber optic, optical fiber, lens, or any other suitable conveyance of light. Examples of a lead extension for use with a lead with electrodes, which can be adapted to also including a light source, can be found in the references cited above.

Although FIG. 1 illustrates that light emitter 126 emitting light from a tip of the lead 102, it will be understood that a light emitter 126 can emit light from the side of the lead 102, as illustrated in FIG. 3, and may emit light around the entire circumference of the lead or only a portion of the circumference as a segmented light emitter 127, as illustrated in FIG. 3. Although FIG. 1 illustrates a single light emitter 126, it will be understood that a lead 102 can have multiple light emitters (e.g., light sources or light emission regions), as illustrated in FIG. 3, which may be the same or may differ in size, shape, orientation, emission frequency, or the like or any combination thereof.

Examples of PBM and PBM/electrical stimulation systems can be found at, for example, U.S. Pat. Nos. 9,415,154; 10,335,607; and 10,814,140; and U.S. patent Applications Publications Nos. 2020/0155854; 2021/0008388; 2021/0008389; 2021/0016111; and 2022/0072329, all of which are incorporated herein by reference in their entireties. Examples of electrical and PBM/electrical stimulation systems with leads that can be used or modified to include the elements described herein are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated herein by reference in their entireties.

Lights sources, such as LEDs or laser diodes, generate heat during operation. In at least some instances, heating can decrease the electrical to optical conversion efficiency of the light source and may also impact the performance of adjacent elements. In addition, operation of the light source may result in heating of adjacent tissue. As described herein, in at least some embodiments, heating of the light source can be monitored. Alternatively or additionally, in at least some embodiments, the light source can be managed to provide safe and efficient operation.

In at least some embodiments, a lead 103, control module 102, or light source 150 (including a light emitter 126 that is a light source) can include a heat monitoring arrangement 170 (FIG. 1) such as a thermocouple, a thermistor, a resistance thermal detector (RTD), infrared sensor, an integrated circuit thermal sensor, or the like or any combination thereof. In at least some embodiments, the heat monitoring arrangement 170 is positioned at or near the light source to estimate a temperature of the light source but can be positioned at or near the tissue or any other suitable element in other embodiments. The heat monitoring arrangement 170 can be used to estimate the temperature of the light source, the lead, the tissue, or any other suitable element or any combination thereof.

As an example, the temperature of the PN junction in a LED or laser diode can be estimated. In at least some embodiments, a thermocouple may be placed in the lead or at a solder terminal of the LED or laser diode to monitor lead or solder temperature. In at least some embodiments, the diode junction temperature can be estimated using a thermal resistance model, for example, T_{J}=(P×R_{th(L-J)}) T_{L}, where T_{J} is the junction temperature, T_{L} is the lead temperature, P is the power dissipation in diode, and R_{th(L-J)}is the thermal resistance between the junction and the lead. In at least some embodiments, if a junction-to-ambient thermal resistance is available, a thermocouple can monitor the ambient temperature (for example, the tissue temperature) and the junction temperature can be estimated as T_{J}=(P×R_{th(A-J)})+T_{A}, where T_{A} is the ambient temperature and R_{th(A-J)} is the junction-to-ambient thermal resistance.

In at least some embodiments, temperature can be estimated or monitored through temperature-dependent characteristics of the light source 150 or other element. In at least some embodiments, one or more of the following can be used to determine or estimate temperature of the LED or laser diode: voltage or current (e.g., the I-V relationship), impedance, the dV/dI curve, a threshold current (for example, detected through thresholding or a linear-fit or 1^{st}, or 2^{nd} derivative of I-V curve), or the like or any combination thereof. Examples of temperature dependence can include, but are not limited to, the V-I curve (FIG. 6), the dV/dI curve (FIG. 7), the threshold current (FIG. 8), impedance (FIG. 9), or the like or any combination thereof. In at least some embodiments, in a current controlled device, the driving current through the LED and laser diode can be selected and the corresponding forward voltage can be measured across the LED or laser diode. (Similarly, in a voltage-controlled device, the driving current through the LED or laser diode can be monitored after selecting the voltage.) In at least some embodiments, the forward voltage on the LED or laser diode may also be assessed through compliance voltage (CV) monitoring. In at least some embodiments, the I-V relationship can be characterized, measured, or assessed as impedance. In at least some embodiments, a series of I-V paired values can be measured within the diode operation range. In at least some embodiments, based on one or more measurements of any of the characteristics of the LED or laser diode, the temperature of the LED or laser diode can be determined or estimated using a look-up table, template matching, fitting a model, or the like or any combination thereof.

As an example of temperature measurement or estimation, a series of burst packets, each burst packet including multiple short pulses, can be performed using the light source 150. The amplitude of the current or voltage applied to the light source 150 is a different value for each of the burst packets and can be the same for all of the pulses in a particular burst packet. (In at least some embodiments, the amplitude can be varied according to a set of planned amplitude step sizes. In at least some embodiments, the variation can include a ramp-up, a wind-down, or both to assess heating up or cooling down.) Measurements of an uncontrolled characteristic, such as voltage, current, impedance, conductance, or the like, can be made for one or more (or all) of the pulses in each burst packet. In at least some embodiments, measurements can be averaged over the pulses in a burst packet when measurement are made for multiple pulses in a burst packet. In at least some embodiments, to estimate the temperature, the measurements or derived metrics can be compared to characterization data through a look-up table, by curve or template matching, by model fitting, or by any other suitable mechanism.

In at least some embodiments, monitoring, estimating, or determining a temperature of tissue that is targeted for PBM or near the lead can be a surrogate for the temperature of the light source (or can be the target for thermal management.) In at least some embodiments, a heat monitoring arrangement 170 (FIG. 3) such as a thermocouple, a thermistor, a resistance thermal detector (RTD), infrared sensor, or an integrated circuit thermal sensor, can be placed at or adjacent the tissue. In addition to temperature, metrics reflecting electrical properties of surrounding tissues (for example, impedance, capacitance, or the like) can be monitored or measured to infer temperature of the tissue. In at least some embodiments, electrodes 134 or electrical sensors, on the lead 103 or using another lead or device, in or near the tissue can monitor or measure these properties of the tissue.

Yet another method for determining or estimating temperature of a light source is to monitor or determine emission efficiency of the light source 150. In at least some embodiment, a light detector 152 (FIG. 2), such as a photodiode, phototransistor, photoresistor, or the like, can be used to measure or monitor light emission or light output of the light source 150. In at least some embodiments, the light output power can be compared to the input power from the electronic subassembly 110 to estimate the amount of heat generated by the light source 150 and may allow estimation of the temperature of the light source. In at least some embodiments, decreasing efficiency (for example, less light output for the same input power) can indicate temperature or a temperature increase. In at least some embodiments, a spectral shift, or other change in the spectrum, of the light output, as observed using the light detector 152, may be indicative of temperature.

In at least some embodiments, a cooling device 172 (FIGS. 2 and 3) disposed near or adjacent to the light source (including a light emitter 126 that is a light source) can be used for temperature management. Examples of cooling devices include, but are not limited to, a heat sink (for example, a block of metal or other thermally conductive material or a reservoir of a phase change material that is solid inside the body but changes phase when heated by the light source 150), a thermoelectric (TE) cooling device (such as a Peltier device), or the like or any combination thereof. In at least some embodiments, a thermoelectric cooling device can be operated by the control module 102 and, for light sources 150 remote from the control module, can use conductors that extend along the lead 103. For example, one channel of the control module 102 can operate the light source 150 and another channel can operate the thermoelectric cooling device. The two channels may be operated in a coordinated manner to manage the temperature of the light source 150.

In at least some embodiments, operation of the light source can be used for temperature management. Heating can be managed through selection of values for delivery parameters such as amplitude (e.g., input current/voltage or charge), pulse width, pulse rate, bursting period, length of period between bursts, duty cycle (for example, a ratio of on time and off time or percentage of on time or off time), duration of a cycle of bursts, length of the period between cycles, the timing/order/sequency of delivery channels, or the like or any combination thereof.

In at least some embodiments, multiple light sources can be operated to provide the photobiomodulation with reduced heat generation, or temperature change, that would be achieved using a single light source. For example, the multiple light sources can be operated in a sequence or a different light source can be activated when one light source reaches a threshold temperature, temperature change, or amount of heat generated. In at least some embodiments, the multiple light sources can be operated using a single delivery channel (using, for example, multiplexing) or multiple delivery channels.

In at least some embodiments, known methods of computational modeling can predict the generation of heat or a heating profile for a set of delivery parameter values and guide the programming of pulse sequences and the duration of operation. In at least some embodiments, a model (2-dimensional model or multi-dimensional model) of radiation intensity vs amplitude, pulse width, duty cycle, pulse rate, or the like or the combination of two or more delivery parameters can provide guidance for programming. For example, selection of a set of delivery parameter values including, for example, amplitude, pulse width, pulse rate, and duty cycle can be used to estimate the heat generation or change in temperature that would be expected during operation of the light source 150 using these delivery parameter values. In at least some embodiments, additional consideration may be given for the presence of a heat sink, cooling device, or any other elements that conductive thermal energy away from the light source 150.

In at least some embodiments, the selection of values for one or more of the delivery parameters during programming of the operation of the light source can be limited based on heat generation. In at least some embodiments, as a program is generated, a programming device can estimate heat generation and prohibit or warn against selections that are likely to result in operational temperatures above a threshold. A program is one or more sets of delivery parameter values, where, for multiple set, photobiomodulation for each set can be delivered simultaneously using multiple channels or can be delivered in any suitable sequence or can be delivered using any combination of simultaneous or sequential delivery.

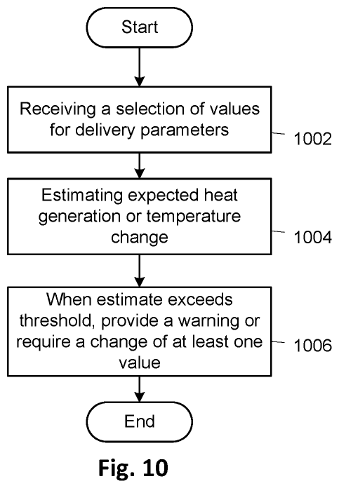

FIG. 10 is a flow chart of one method for generating a delivery program. In step 1002, a selection of values for delivery parameters are received. In at least some embodiments, the selection of the value for a delivery parameter can be limited by expected amount, speed, or time of any one, or any combination, of heat generation or temperature change arising from the selection of that value and any previously selected values. In step 1004, after the selection of all values, the expected amount, speed, or time of any one, or any combination, of heat generation or temperature change is estimated. In step 1006, when the estimate exceeds a threshold, the system can provide a warning or recommendation (for example, a change to one or more parameters) to the programmer, can require the programmer to alter at least one of the selected values, or any other corrective action, or any combination thereof.

In at least some embodiments, one or more thresholds can be used to classify thermal conditions. In at least some embodiments, a modulation function can be used to predict and suggest changes to the program settings or device operation for thermal management, as described above. In at least some embodiments, in an open loop setting or other setting, a dedicated channel of the control module 102 may be used concurrently to provide cooling of the light source 150. In at least some embodiments, in a semi-closed loop setting or other setting, a warning signal or messages may be provided to notify a user about the thermal conditions and request action to modify the light delivery parameters or the cooling settings. In at least some embodiments, a suggestion of a change in delivery program, one or more delivery parameters, or operation can be suggested to the user by the system. In at least some embodiments, in a closed loop setting or other setting, a threshold can trigger a change in operation of the control module 102 (for example, halting light generation or switching between light delivery programs or adjusting one or more delivery parameters) or a change in thermoelectrical cooling settings (for example, turning on or off a cooling device or increasing/decreasing operation of the cooling device). In at least some embodiments, thermal management can be combined with battery efficiency management to select delivery parameters that provide good thermal and power management.

In at least some embodiments, a program can be designated as “warm” and “cool” (or any other similar designation) where the designation relates to the relative amount, speed, or time of any one, or any combination of, heat generated, temperature, or temperature change that could be obtained, during operation of the program. In at least some embodiments, a user can designate a program as “warm” or “cool”. In at least some embodiments, a device, such as a clinician programmer, can evaluate the program and can designate the program as “warm” or “cool”. Such evaluation may be based on any suitable methodology including, but not limited to, comparison to threshold values for one or more delivery parameters or combinations of delivery parameters, using a heat generation or temperature change algorithm, analytical or mathematical model, or the like or any combination thereof.

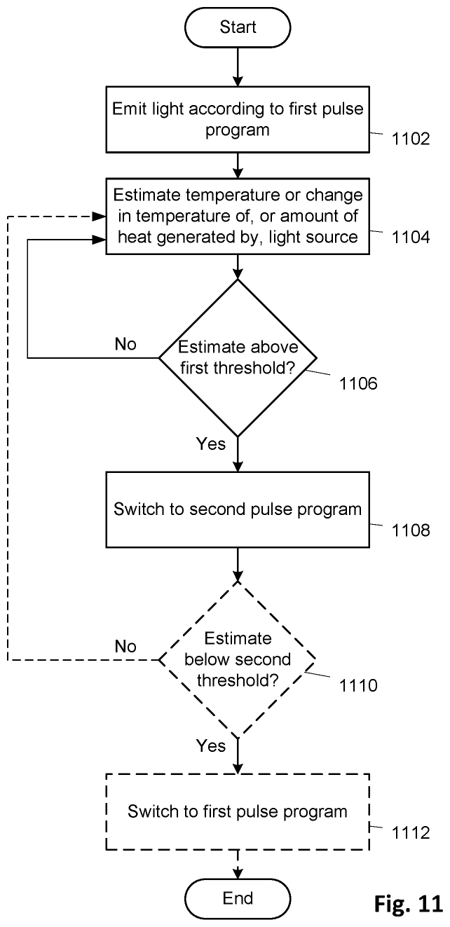

During operation, the control module, another device, or a user can switch between programs. In at least some embodiments, the control module 102 (or other device or a user) can switch between a “warm” program and a “cool” program based on a detected thermal condition (for example, an estimated temperature, temperature change, or amount of heat generated.) For example, in at least some embodiments, light is emitted according to a first delivery program, which is designated a “warm” program (or the like), in step 1102 of FIG. 11. In step 1104, a temperature or change in temperature of, or amount of heat generated by, the light source 150 (or any other suitable indicator) can be estimated. For example, any of the methods and arrangements, discussed above, for estimating or measuring an amount, speed, or time of any one, or any combination, of temperature, change in temperature, or heat generation; any other suitable indicator; or the like or any combination thereof can be used. In steps 1106 and 1108, the control module 102 (or other device or a user) can switch from a “warm” program (the first delivery program) to a “cool” program (a second delivery program) when the measured or estimated amount, speed, or time of ay one, or any combination, of temperature, change in temperature, or heat generation; any other suitable indicator; or the like or any combination thereof is above a second threshold. In at least some embodiments, the switch between programs can be automatic. In at least some embodiments, the control module 102 can require a user to confirm the switch through a user device, such as a remote control, clinician programmer, smart phone or tablet, or the like, before the switch between programs is performed.

Optionally, in steps 1110 and 1112, the control module 102 (or other device or a user) can switch from the “cool” program to the “warm” program (or a different “warm” program) when the measured or estimated amount, speed, or time of any one, or any combination, of temperature, change in temperature, or heat generation; any other suitable indicator; or the like or any combination thereof is below a second threshold. (The first and second thresholds can be the same or different.) In at least some embodiments, the switch between programs can be automatic. In at least some embodiments, the control module 102 can require a user to confirm the switch through a user device, such as a remote control, clinician programmer, smart phone or tablet, or the like, before the switch between programs is performed. It will be recognized that the procedure can continuously repeat until light is no longer emitted according to any delivery program.

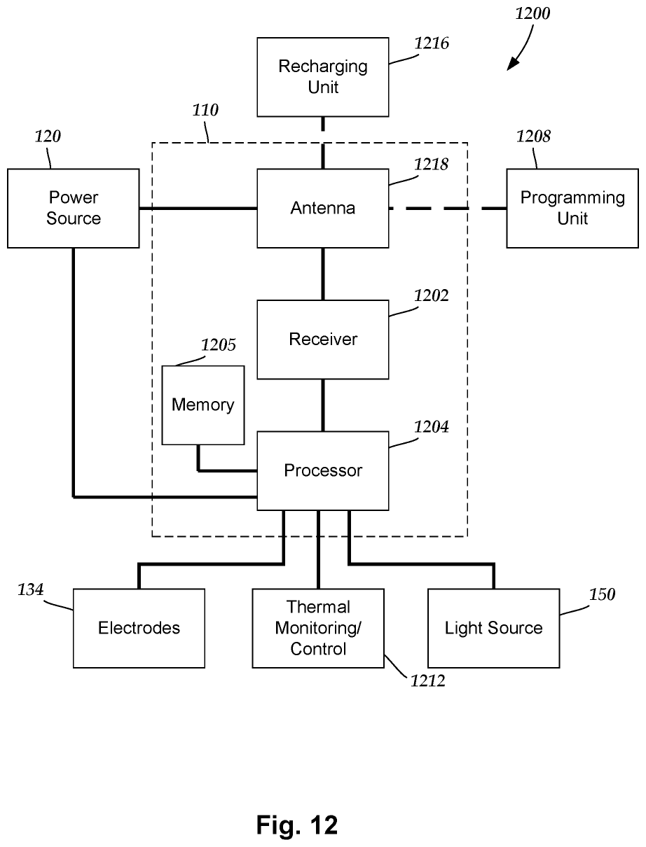

FIG. 12 is a schematic overview of one embodiment of components of an PBM or PBM/electrical stimulation system 1200 including an electronic subassembly 110 disposed within a control module 102 (for example, an implantable pulse generator). It will be understood that the PBM or PBM/electrical stimulation system can include more, fewer, or different components and can have a variety of different configurations including those configurations disclosed in the stimulator references cited herein.

In at least some embodiments, selected components (for example, a power source 120, an antenna 1218, a receiver 1202, a processor 1204, and a memory 1205) of the PBM or PBM/electrical stimulation system can be positioned on one or more circuit boards or similar carriers within a sealed housing of a control module 102. Any suitable processor 1204 can be used and can be as simple as an electronic device that, for example, produces signals to direct or generate PBM or PBM/electrical stimulation at a regular interval or the processor can be capable of receiving and interpreting instructions from an external programming unit 1208 that, for example, allows modification of delivery parameters or characteristics.

The processor 1204 is generally included to control the timing and other characteristics of the PBM or PBM/electrical stimulation system. For example, the processor 1204 can, if desired, control one or more of the timing, pulse frequency, amplitude, and duration of the PBM or PBM/electrical stimulation. In addition, the processor 1204 can select one or more of the electrodes 134 to provide electrical stimulation, if desired. In some embodiments, the processor 1204 selects which of the electrode(s) are cathodes and which electrode(s) are anodes. The processor 1204 includes or is coupled to thermal monitoring or thermal control elements 1212 (or elements that provide both), as described above.

Any suitable memory 1205 can be used. The memory 1205 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, magnetic storage devices, or any other medium which can be used to store the desired information and which can be accessed by a processor.

The processor 1204 is coupled to a light source 150. Any suitable light source can be used including, but not limited to, LEDs, OLEDs, LETs, OLETs, laser diodes, VCSELs, lamps, light bulbs, or the like or any combination thereof. In at least some embodiments, the PBM or PBM/electrical stimulation system may include multiple light sources. In at least some embodiments, each of the multiple light sources may emit light having a same or different wavelength or a same or different wavelength range. Any suitable wavelength or wavelength range can be used including, but not limited to, visible, near infrared, and ultraviolet wavelengths or wavelength ranges. A wavelength or wavelength range of a light source may be selected to obtain a specific therapeutic, chemical, or biological effect.

Any power source 120 can be used including, for example, a battery such as a primary battery or a rechargeable battery. Examples of other power sources include super capacitors, nuclear or atomic batteries, fuel cells, mechanical resonators, infrared collectors, flexural powered energy sources, thermally-powered energy sources, bioenergy power sources, bioelectric cells, osmotic pressure pumps, and the like. As another alternative, power can be supplied by an external power source through inductive coupling via an antenna 1218 or a secondary antenna. The external power source can be in a device that is mounted on the skin of the user or in a unit that is provided near the user on a permanent or periodic basis. In at least some embodiments, if the power source 1212 is a rechargeable battery, the battery may be recharged using the antenna 1218 and a recharging unit 1216. In some embodiments, power can be provided to the battery for recharging by inductively coupling the battery to the external recharging unit 1216.

In at least some embodiments, the processor 1204 is coupled to a receiver 1202 which, in turn, is coupled to an antenna 1218. This allows the processor 1204 to receive instructions from an external source, such as programming unit 1208, to, for example, direct the delivery parameters and characteristics. The signals sent to the processor 1204 via the antenna 1218 and the receiver 1202 can be used to modify or otherwise direct the operation of the PBM or PBM/electrical stimulation system. For example, the signals may be used to modify the characteristics or delivery parameters of the PBM or PBM/electrical stimulation system. The signals may also direct the PBM or PBM/electrical stimulation system 1200 to cease operation, to start operation, to start charging the battery, or to stop charging the battery. In other embodiments, the PBM or PBM/electrical stimulation system does not include the antenna 1218 or receiver 1202 and the processor 1204 operates as initially programmed.

In at least some embodiments, the antenna 1218 is capable of receiving signals (e.g., RF signals) from an external programming unit 1208 (such as a clinician programmer or patient remote control or any other device) which can be programmed by a user, a clinician, or other individual. The programming unit 1208 can be any unit that can provide information or instructions to the PBM or PBM/electrical stimulation system 1200. In at least some embodiments, the programming unit 1208 can provide signals or information to the processor 1204 via a wireless or wired connection. One example of a suitable programming unit is a clinician programmer or other computer operated by a clinician or other user to select, set, or program delivery parameters for the PBM or PBM/electrical stimulation system. Another example of the programming unit 1208 is a remote control such as, for example, a device that is worn on the skin of the user or can be carried by the user and can have a form similar to a pager, cellular phone, or remote control, if desired. In at least some embodiments, a remote control used by a patient may have fewer options or capabilities for altering delivery parameters than a clinician programmer.

Optionally, the PBM or PBM/electrical stimulation system 1200 may include a transmitter (not shown) coupled to the processor 1204 and the antenna 1218 for transmitting signals back to the programming unit 1208 or another unit capable of receiving the signals. For example, the PBM or PBM/electrical stimulation system 1200 may transmit signals indicating whether the PBM or PBM/electrical stimulation system 1200 is operating properly or not or indicating when the battery needs to be charged or the level of charge remaining in the battery. The processor 1204 may also be capable of transmitting information about the delivery parameters or characteristics so that a user or clinician can determine or verify the delivery parameters or characteristics.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, at least one process may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, in the cloud or other non-local site, or any other medium which can be used to store the desired information and which can be accessed by a computing device.

A system can include one or more processors that can perform the methods (in whole or in part) described above. In at least some embodiments, some or all of the method may be performed using one or more non-local processor(s) (for example, processors in another device or in the cloud.) The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process. In at least some embodiments, the processor may include more than one processor.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A method for photobiomodulation of tissue, the method comprising:
emitting light from a lead implanted in the tissue using an implanted light source according to a first delivery program;
repeatedly estimating an amount, speed, or time of any one, or any combination of, a temperature or a temperature change of, or amount of heat generated by, the implanted light source or repeatedly estimating an amount, speed, or time of any one, or any combination of, a temperature or a temperature change of tissue receiving the emitted light; and
when the estimate exceeds a first threshold value and the light is emitted according to the first delivery program, emitting light from the implanted lead using the implanted light source according to a second delivery program, wherein the second delivery program results in lower heat generation by the implanted light source over a period of time than the first delivery program.

2. The method of claim 1, further comprising, when the estimate falls below a second threshold value and the light is emitted according to the second delivery program, emitting light from the implanted lead using the implanted light source according to the first delivery program.

3. The method of claim 2, wherein the first threshold value and the second threshold value are different.

4. The method of claim 1, wherein the estimating comprises making a measurement to estimate the amount, speed, or time of any one, or any combination, of the temperature, temperature change, or amount of heat generated.

5. The method of claim 4, wherein making the measurement comprises making the measurement regularly with a specified periodicity.

6. The method of claim 4, wherein making the measurement comprises making a temperature measurement using a thermocouple, a thermistor, a resistance thermal detector (RTD), infrared sensor, or an integrated circuit thermal sensor.

7. The method of claim 4, wherein making the measurement comprises making an electrical measurement of the light source.

8. The method of claim 7, wherein making the electrical measurement comprises making a measurement of current, voltage, or impedance, or of a change in current, voltage, or impedance, of the light source.

9. The method of claim 4, wherein making the measurement comprises making a measurement of the light emitted by the light source.

10. The method of claim 1, wherein emitting light from the implanted lead using the implanted light source according to the second delivery program comprises
when the estimate exceeds a first threshold value and the light is emitted according to the first delivery program, requesting user confirmation to switch to the second delivery program, and
after receiving the user confirmation, emitting light from the implanted lead using the implanted light source according to the second delivery program.

11. The method of claim 10, further comprising
when the estimate falls below a second threshold value and the light is emitted according to the second delivery program, requesting user confirmation to switch to the first delivery program; and
after receiving the user confirmation, emitting light from the implanted lead using the implanted light source according to the first delivery program.

12. The method of claim 10, wherein the estimating comprises making a measurement to estimate the amount, speed, or time of any one, or any combination, of the temperature, temperature change, or amount of heat generated.

13. The method of claim 12, wherein making the measurement comprises making the measurement regularly with a specified periodicity.

14. The method of claim 12, wherein making the measurement comprises making a temperature measurement using a thermocouple, a thermistor, a resistance thermal detector (RTD), infrared sensor, or an integrated circuit thermal sensor.

15. The method of claim 12, wherein making the measurement comprises making an electrical measurement of the light source.

16. A system for photobiomodulation of tissue, the system comprising:
a light source;
an implantable lead comprising a distal region and a light emitter disposed along the distal region, wherein the light emitter is either the light source or coupled to the light source by at least one optical waveguide so that light from the light source is emitted from the light emitter; and
an implantable control module coupled to the light source for directing the light source to generated light, the implantable control module comprises a memory storing instructions and a processor configured to execute the instructions, the instructions comprising the method of claim 1.

17. The system of claim 16, wherein the instructions further comprise
when the estimate falls below a second threshold value and the light is emitted according to the second delivery program, emitting light from the implanted lead using the implanted light source according to the first delivery program.

18. A photobiomodulation system, comprising:
a programmer comprising
a processor configured for programming of an implantable control unit for generating a delivery program for an implanted light source, the processor configured to perform actions comprising:
receiving a selection of a value for each of a plurality of delivery parameters, wherein the selection of the value for each of the delivery parameters is limited by expected heat generation or temperature change arising from the selection of the value and any previously selected values; and
after the selection of all of the values, estimating expected heat generation and, when the expected heat generation or temperature change exceeds a threshold either i) providing a warning or recommendation to a programmer or ii) requiring the programmer to alter at least one of the selected values.

19. A photobiomodulation system, comprising:
an implantable light source;
an implantable lead comprising a distal region and a light emitter disposed along the distal region, wherein the light emitter is either the implantable light source or coupled to the implantable light source by at least one optical waveguide so that light from the implantable light source is emitted from the light emitter into tissue;
a thermoelectric cooling device coupled to the implantable light source and configured for removing heat generated by the implantable light source; and
an implantable control module coupled to the implantable light source for directing the implantable light source to generate light and coupled to the thermoelectric cooling device for directing the thermoelectric cooling device to remove heat generated by the implantable light source.

20. The photobiomodulation system of claim 19, wherein the implantable light source and the thermoelectric cooling device are disposed in the implantable lead.
